(19) United States
(12) Patent Application Publication
(10) Pub. No.: US 2020/0060651 A1
(43) Pub. Date: Feb. 27, 2020

(54) ARRAY-TYPE ULTRASONIC VIBRATOR, ULTRASONIC PROBE, ULTRASONIC CATHETER, HAND-HELD SURGICAL INSTRUMENT, AND MEDICAL APPARATUS

(71) Applicant: SONY CORPORATION, TOKYO (JP)

(72) Inventors: RUI MORIMOTO, KANAGAWA (JP); AKIHIRO NAKATA, KANAGAWA (JP)

(21) Appl. No.: 16/462,818

(22) PCT Filed: Oct. 13, 2017
(86) PCT No.: PCT/JP2017/037211

§ 371 Date: May 21, 2019

(30) Foreign Application Priority Data
Dec. 20, 2016 (JP) 2016-246688

(51) Int. Cl. A61B 8/00; A61B 8/12

(52) U.S. Cl. CPC A61B 8/4488; A61B 8/445; A61B 8/12

(57) ABSTRACT

[Object] To provide an array-type ultrasonic vibrator, which is excellent in productivity and practicability and has a two-dimensional array structure capable of improving a focusing property of an ultrasonic beam in a slice direction, an ultrasonic probe, an ultrasonic catheter, a hand-held surgical instrument, and a medical apparatus. [Solving Means] An array-type ultrasonic vibrator according to an embodiment of the present technology includes a vibrator array and a resistor. The vibrator array is a vibrator array in which ultrasonic vibrator elements constitute a two-dimensional array including a plurality of element rows in which a plurality of ultrasonic vibrator elements are arranged in a slice direction. The resistor is electrically connected between a pair of arbitrary ultrasonic vibrator elements in the element row.

### TECHNICAL FIELD

The present technology relates to an array-type ultrasonic vibrator, an ultrasonic probe, an ultrasonic catheter, a hand-held surgical instrument, and a medical apparatus, which can be used for generating an ultrasonic diagnosis image.

### BACKGROUND ART

In ultrasonic imaging for the use in the medical field and the like, an ultrasonic probe including an ultrasonic vibrator array radiates an ultrasonic wave to an object to be observed. The ultrasonic probe detects a reflected wave thereof. An ultrasonic image of the object to be observed is thus generated. The ultrasonic imaging makes a biological tissue visible from outside. The ultrasonic imaging is suitable for grasping a blood stream and the position and shape of a tumor or finding a nerve associated with a blood vessel, for example.

An ultrasonic beam width in a slice direction (depth direction of the ultrasonic image) in the ultrasonic imaging acts on slice resolution and contrast. Therefore, in recent years, reduction of this beam width has been developed. For example, in a one-dimensional array in which ultrasonic vibrators are arranged in one row, the beam width in the slice direction can be made narrower by focusing an ultrasonic beam through an acoustic lens.

On the other hand, in a two-dimensional array in which the ultrasonic vibrators are arranged in a flat state, technologies such as apodization and phase adjustment are used for improving a focusing property of the ultrasonic beam in the slice direction. The apodization is a technology of reducing the outputs of the vibrators at the ends of the two-dimensional array. In the apodization, the focusing property of the beam is improved by reducing components of side lobes (ultrasonic waves travelling in a direction deviated from a main radiation direction). Further, in the phase adjustment, the focusing property of the beam is improved by voluntarily adjusting a phase difference between the vibrators (e.g., Patent Literature 1).

In addition, there is also well known a structure called Hanafy lens. In this structure, the focusing property of the beam is improved by making the thickness of the vibrators different to thereby form the entire two-dimensional array in the lens-like shape.

### CITATION LIST

### Patent Literature

Patent Literature 1: Japanese Patent Application Laid-open No. 2014-097157

### DISCLOSURE OF INVENTION

### Technical Problem

However, in order to realize the above-mentioned apodization and phase adjustment, it is necessary to individually control the output of each of the ultrasonic vibrators, and the number of wirings to be connected to the respective vibrator increases. Therefore, the manufacture complication and the manufacture cost increase. Further, it becomes necessary to introduce a multiplexer and the like into the ultrasonic probe, and the structure of the ultrasonic probe is complicated.

Also in a case where the Hanafy lens structure is formed, it becomes necessary to machine the vibrators. In particular, in a case where the vibrators are thin, it is difficult to make the thickness of the vibrators different and it is not easy to cause the vibrators to have a higher frequency and a smaller height.

In view of the above-mentioned circumstances, it is an object of the present technology to provide an array-type ultrasonic vibrator, which is excellent in productivity and practicability and has a two-dimensional array structure capable of improving a focusing property of an ultrasonic beam in a slice direction, an ultrasonic probe, an ultrasonic catheter, a hand-held surgical instrument, and a medical apparatus.

### Solution to Problem

In order to accomplish the above-mentioned object, an array-type ultrasonic vibrator according to an embodiment of the present technology includes a vibrator array and a resistor.

The vibrator array is a vibrator array in which ultrasonic vibrator elements constitute a two-dimensional array including a plurality of element rows in which a plurality of ultrasonic vibrator elements are arranged in a slice direction.

The resistor is electrically connected between a pair of arbitrary ultrasonic vibrator elements in the element row.

In accordance with this configuration, when the wiring connected to the element row is supplied with a driving signal of the ultrasonic vibrator element, the driving signal is attenuated by the resistor and the intensity of the driving signal supplied to the ultrasonic vibrator element at the end portion of the element row is thus lowered, and the output of the ultrasonic wave radiated from the ultrasonic vibrator element at the end portion of the element row becomes smaller (apodization). In this manner, the beam width of the ultrasonic beam in the slice direction can be reduced, and the slice resolution and contrast of the ultrasonic diagnosis image can be enhanced. Further, it is only necessary to connect the single wiring to the single element row and it is unnecessary to connect the wiring to each of the ultrasonic vibrator elements. Therefore, the number of wirings can be greatly reduced.

The resistor may be electrically connected between all ultrasonic vibration elements in the element row.

By the resistor being arranged between all the ultrasonic vibration elements, the outputs of the ultrasonic waves radiated from the respective ultrasonic vibrator elements that constitute the element row can be made slightly different from one another.

The array-type ultrasonic vibrator may further include a grounding resistor connected between an ultrasonic vibration element at an end of the element row and a ground.

By providing the grounding resistor, an escape path for electric charges can be ensured at the end portion of the element row and electric charges can be prevented from being accumulated at that end portion.

The vibrator array may include a substrate that supports the ultrasonic vibrator elements, and the resistor may be implemented on a surface of the substrate or inside the substrate.

In accordance with this configuration, the vibrator array and the resistor can be configured integrally with each other, and the size and height of the array-type ultrasonic vibrator can be reduced.

The ultrasonic vibrator elements may be connected to a wiring for driving the ultrasonic vibrator elements in each of the element rows.

In accordance with this configuration, the outputs of the ultrasonic waves emitted from the ultrasonic elements in each element row can be adjusted. It should be noted that the outputs of the ultrasonic waves emitted from the ultrasonic elements are adjusted by the resistor in each element row.

In order to accomplish the above-mentioned object, an ultrasonic catheter according to an embodiment of the present technology includes an array-type ultrasonic vibrator.

The array-type ultrasonic vibrator includes a vibrator array in which ultrasonic vibrator elements constitute a two-dimensional array including a plurality of element rows in which a plurality of ultrasonic vibrator elements are arranged in a slice direction, and a resistor electrically connected between a pair of arbitrary ultrasonic vibration elements in the element row.

In order to accomplish the above-mentioned object, a hand-held instrument according to an embodiment of the present technology includes an array-type ultrasonic vibrator.

The array-type ultrasonic vibrator includes a vibrator array in which ultrasonic vibrator elements constitute a two-dimensional array including a plurality of element rows in which a plurality of ultrasonic vibrator elements are arranged in a slice direction, and a resistor electrically connected between a pair of arbitrary ultrasonic vibration elements in the element row.

In order to accomplish the above-mentioned object, a medical apparatus according to an embodiment of the present technology includes an array-type ultrasonic vibrator and a position sensor.

The array-type ultrasonic vibrator includes a vibrator array in which ultrasonic vibrator elements constitute a two-dimensional array including a plurality of element rows in which a plurality of ultrasonic vibrator elements are arranged in a slice direction, and a resistor electrically connected between a pair of arbitrary ultrasonic vibration elements in the element row.

The position sensor detects a position of the array-type ultrasonic vibrator.

In the medical apparatus, an ultrasonic volume image may be generated on the basis of outputs of the array-type ultrasonic vibrator and the position sensor.

In the array-type ultrasonic vibrator, in each of the element rows, a total resistance value of the resistor and the grounding resistor may be larger than a resistance value of a signal wiring that connects the ultrasonic vibrator element to a driving power supply.

By setting the total resistance value of the resistor and the grounding resistor to be larger than the resistance value of the signal wiring, a voltage drop in each vibrator element can be reduced.

In the array-type ultrasonic vibrator, in each of the element rows, provided that a frequency of a driving voltage of the ultrasonic vibrator element is f [Hz], a product of a total resistance value of the resistor and a total value of capacitance of the ultrasonic vibrator elements may be smaller than ½ f.

By setting the product of the total resistance value of the resistor and the total value of capacitance of the ultrasonic vibrator elements to be smaller than ½ f, an RC delay (phase offset) of the ultrasonic wave emitted from each ultrasonic vibrator element can be prevented.

In the array-type ultrasonic vibrator, in each of the element rows, a resistance value of the grounding resistor may be smaller than a total resistance value of the resistor.

By setting the resistance value of the grounding resistor to be smaller than the total resistance value of the resistor, the dynamic range of the ultrasonic waves in the element row can be made larger.

### Advantageous Effects of Invention

As described above, in accordance with the present technology, it is possible to provide an array-type ultrasonic vibrator, which is excellent in productivity and practicability and has a two-dimensional array structure capable of improving a focusing property of an ultrasonic beam in a slice direction, an ultrasonic probe, an ultrasonic catheter, a hand-held surgical instrument, and a medical apparatus. It should be noted that the effects described here are not necessarily limitative and any effect described in the present disclosure may be provided.

### BRIEF DESCRIPTION OF DRAWINGS

FIG. 1 A perspective view of an array-type ultrasonic vibrator according to an embodiment of the present technology.

FIG. 2 A perspective view of a partial configuration of the array-type ultrasonic vibrator.

FIG. 3 A plan view of vibrator elements of the array-type ultrasonic vibrator.

FIG. 4 A cross-sectional view of the array-type ultrasonic vibrator.

FIG. 5 A schematic diagram showing an array of the vibrator elements of the array-type ultrasonic vibrator.

FIG. 6 A schematic diagram showing an electric connection relationship of an element row of the array-type ultrasonic vibrator.

FIG. 7 A schematic diagram showing an operation of the element row of the array-type ultrasonic vibrator.

FIG. 8 A schematic diagram showing resistors of the array-type ultrasonic vibrator.

FIG. 9 A schematic diagram showing independent wirings of the array-type ultrasonic vibrator.

FIG. 10 A schematic diagram showing an electric connection relationship of the element row of the array-type ultrasonic vibrator.

FIG. 11 A schematic diagram showing an electric connection relationship of the element row of the array-type ultrasonic vibrator.

FIG. 12 A cross-sectional view of an ultrasonic probe of the array-type ultrasonic vibrator.

FIG. 13 A cross-sectional view of the ultrasonic probe of the array-type ultrasonic vibrator.

FIG. 14 A graph showing a beam profile of the ultrasonic probe of the array-type ultrasonic vibrator.

FIG. 15 A schematic diagram of an ultrasonic catheter including the array-type ultrasonic vibrator according to the embodiment of the present technology.

FIG. 16 A graph showing a beam profile of the ultrasonic catheter of the array-type ultrasonic vibrator.

FIG. 17 A schematic diagram of a surgical instrument of the array-type ultrasonic vibrator according to the embodiment of the present technology.

FIG. 18 A schematic diagram showing an electric circuit configuration of each element array of the array-type ultrasonic vibrator according to the embodiment of the present technology.

FIG. 19 A graph showing percentage of resistance values of resistors 133 and a grounding resistor in the array-type ultrasonic vibrator.

FIG. 20 A graph showing simulation results of a voltage time history waveform with each of the vibrator elements in the element row of the array-type ultrasonic vibrator.

FIG. 21 A graph showing simulation results of the voltage time history waveform with each of the vibrator elements in the element row of the array-type ultrasonic vibrator.

FIG. 22 A graph showing simulation results of the voltage time history waveform with each of the vibrator elements in the element row of the array-type ultrasonic vibrator.

FIG. 23 A graph showing simulation results of the voltage time history waveform with each of the vibrator elements in the element row of the array-type ultrasonic vibrator.

FIG. 24 A graph showing simulation results of the voltage time history waveform with each of the vibrator elements in the element row of the array-type ultrasonic vibrator.

FIG. 25 Simulation results of a sound pressure beam profile of the array-type ultrasonic vibrator (opening width: 5 mm).

FIG. 26 A graph showing a beam width with the sound pressure beam profile shown in FIG. 25.

FIG. 27 A graph showing a dynamic range with the sound pressure beam profile shown in FIG. 25.

FIG. 28 Simulation results of a sound pressure beam profile of the array-type ultrasonic vibrator (opening width: 2 mm).

FIG. 29 A graph showing a beam width with the sound pressure beam profile shown in FIG. 28.

FIG. 30 A graph showing a dynamic range with the sound pressure beam profile shown in FIG. 28.

FIG. 31 A graph showing an apodization intensity distribution in the element row of the array-type ultrasonic vibrator.

FIG. 32 A schematic diagram showing a structure of the array-type ultrasonic vibrator.

FIG. 33 A graph showing a hamming window function.

FIG. 34 Simulation results of a sound pressure beam profile of the array-type ultrasonic vibrator (opening width: 5 mm).

FIG. 35 A graph showing a beam width with the sound pressure beam profile shown in FIG. 34.

FIG. 36 A graph showing a dynamic range with the sound pressure beam profile shown in FIG. 34.

FIG. 37 Simulation results of a sound pressure beam profile of the array-type ultrasonic vibrator (opening width: 2 mm).

FIG. 38 A graph showing a beam width with the sound pressure beam profile shown in FIG. 37.

FIG. 39 A graph showing a dynamic range with the sound pressure beam profile shown in FIG. 37.

### MODE(S) FOR CARRYING OUT THE INVENTION

An array-type ultrasonic vibrator according to this embodiment will be described.

### [Configuration of Array-Type Ultrasonic Vibrator]

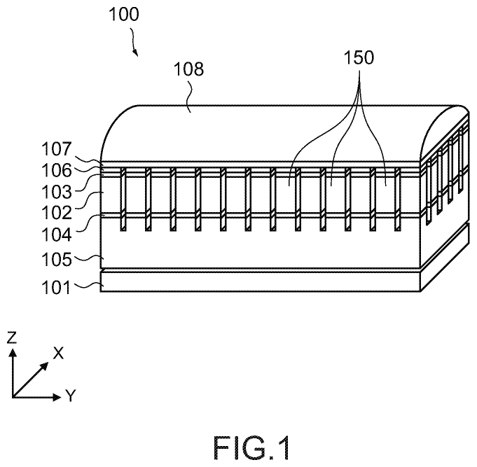

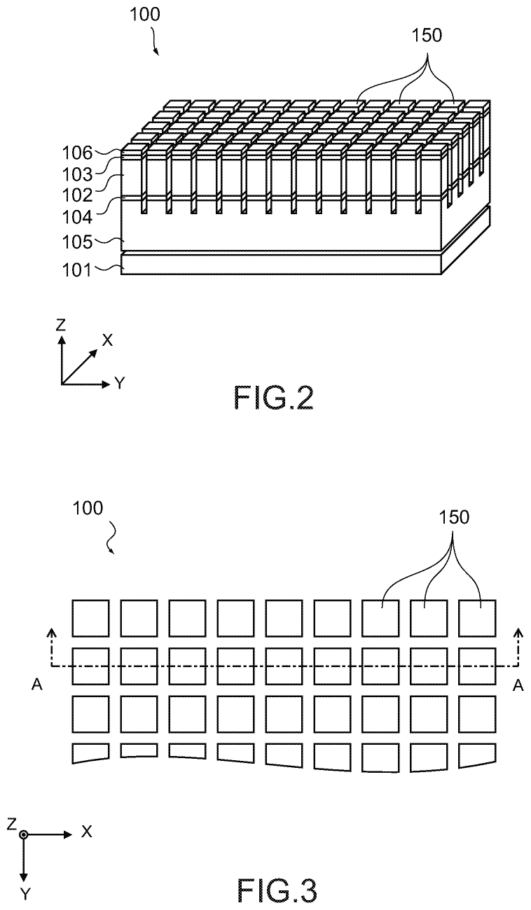

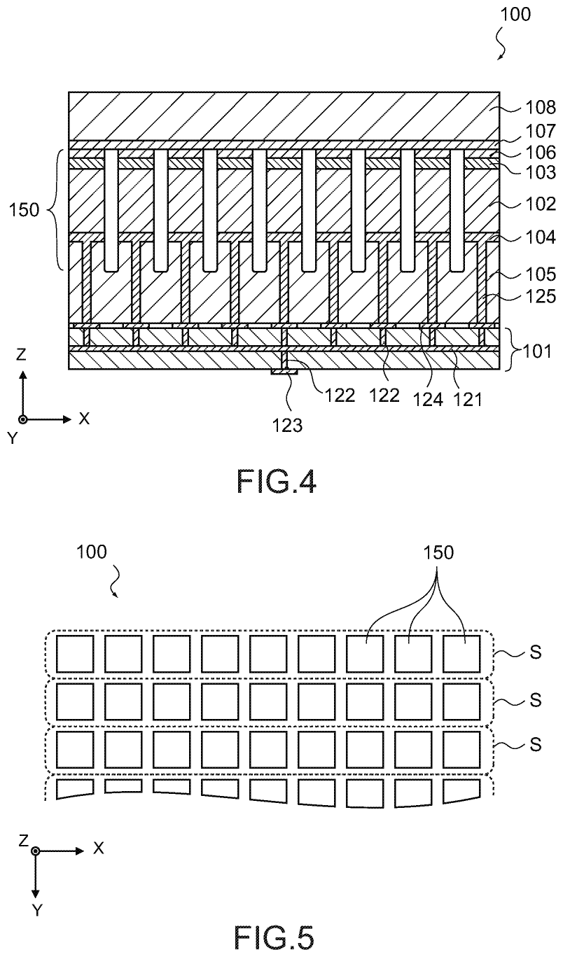

FIG. 1 is a perspective view of an array-type ultrasonic vibrator 100 according to this embodiment. FIG. 2 is a perspective view of a partial configuration of the array-type ultrasonic vibrator 100. FIG. 3 is a plan view of a partial configuration of the array-type ultrasonic vibrator 100. FIG. 4 is a cross-sectional view of the array-type ultrasonic vibrator 100, which is a cross-sectional view taken along the A-A line of FIG. 3. In each figure, three directions orthogonal to one another are denoted by an X direction, a Y direction, and a Z direction.

As shown in FIG. 4, the array-type ultrasonic vibrator 100 includes a substrate 101, piezoelectric layers 102, upper electrode layers 103, lower electrode layers 104, backing layers 105, acoustic matching lenses 106, an acoustic matching lens 107, and an acoustic lens 108.

The piezoelectric layers 102, the upper electrode layers 103, the acoustic matching lenses 106, the lower electrode layers 104, and the backing layers 105 are partially separated from each other, each of which constitutes vibrator elements 150. That is, the array-type ultrasonic vibrator 100 is an array of the vibrator elements 150. It should be noted that although the number of vibrator elements 150 are different between FIGS. 2 and 3, illustration of a predetermined number of vibrator elements 150 is omitted in FIG. 2.

The substrate 101 is a wiring board such as a rigid printed board and a flexible printed circuits (FPC) board including glass epoxy and the like. The substrate 101 supports and electrically connects the vibrator elements 150. The substrate 101 is provided with a substrate built-in resistor 121, wirings 122, an independent wiring 123, and a pad 124.

The pad 124 is provided on the surface of the substrate 101 and each of the vibrator elements 150 is electrically connected thereto. The wirings 122 electrically connect the pad 124 to the substrate built-in resistor 121. Each of the vibrator elements 150 is connected to the substrate built-in resistor 121 via the wirings 122 and the pad 124. The independent wiring 123 is electrically connected to the substrate built-in resistor 121. Electric connection of each of the vibrator elements 150 will be described later.

The piezoelectric layers 102 include a piezoelectric material such as lead zirconate titanate (PZT). The piezoelectric layers 102 are provided between the lower electrode layers 104 and the upper electrode layers 103. When a voltage is applied between the lower electrode layers 104 and the upper electrode layers 103, vibrations due to an inverse piezoelectric effect are generated and ultrasonic waves are generated. Further, when reflected waves from an object to be diagnosed enters the piezoelectric layers 102, polarization due to the piezoelectric effect occurs. Although the size of the piezoelectric layer 102 is not particularly limited, 250 μm square, for example, can be set.

The upper electrode layers 103 are provided on the piezoelectric layers 102. The upper electrode layers 103 include an electrically conductive material. The upper electrode layers 103 are metal films deposited by plating, sputtering, or the like, for example. It should be noted that the upper electrode layers 103 may be separated for each of the vibrator elements 150 as shown in FIG. 4 and do not need to be separated.

The lower electrode layers 104 are provided on the backing layers 105. The lower electrode layers 104 include an electrically conductive material. The lower electrode layers 104 are metal films deposited by plating, sputtering, or the like, for example. The lower electrode layers 104 are electrically connected to the substrate 101 via wirings 125.

The backing layers 105 are provided on the substrate 101 and absorb unnecessary vibrations of the vibrator elements 150. The backing layers 105 generally include a material in which a filler and a synthetic resin are mixed and the like. In the backing layers 105, the wirings 125 that connect the lower electrode layers 104 to the pad 124 are provided.

The acoustic matching lenses 106 and the acoustic matching lens 107 reduce an acoustic impedance difference between the object to be diagnosed and the vibrator elements 150 and prevent reflection of ultrasonic waves from being reflected to the object to be diagnosed. The acoustic matching lenses 106 include a synthetic resin and a ceramic material. As shown in FIG. 4, the acoustic matching lenses 106 can be separated for each of the vibrator elements 150 and the acoustic matching lens 107 can be set not to be separated, though not limited thereto.

The acoustic lens 108 is brought into contact with the object to be diagnosed. The acoustic lens 108 focuses ultrasonic waves generated in the piezoelectric layers 102. The acoustic lens 108 includes a silicone rubber and the like, for example, and the size and shape thereof are not particularly limited.

### [Regarding Array of Vibrator Elements]

As shown in FIGS. 2 and 3, the vibrator elements 150 are arranged in two directions of the X direction and the Y direction as viewed in a thickness direction (Z direction) of the vibrator elements 150.

A shorter side direction (X direction) of the array-type ultrasonic vibrator 100 will be referred to as a slice direction (or elevation direction) and the resolution in that direction is equivalent to a resolution in a depth direction in the ultrasonic diagnosis image. The number of vibrator elements 150 in the slice direction is not particularly limited and only needs to be plural.

A longer side direction (Y direction) of the array-type ultrasonic vibrator 100 will be referred to as an azimuth direction and the resolution in that direction is equivalent to a resolution of an azimuth-based direction in the ultrasonic diagnosis image. The number of vibrator elements 150 in the azimuth direction is not particularly limited and only needs to be plural.

It should be noted that a resolution in the thickness direction (Z direction) of the vibrator elements 150 is equivalent to a resolution in the distance direction in the ultrasonic diagnosis image.

The beam width of the ultrasonic beam in the slice direction acts on the slice resolution and contrast of the ultrasonic diagnosis image. Therefore, it is favorable that this beam width is smaller. In a one-dimensional array-type ultrasonic vibrators in which vibrator elements are arranged in one row in the azimuth direction, the beam width in the slice direction can be reduced by using an acoustic lens.

On the other hand, in a two-dimensional array-type ultrasonic vibrator in which a plurality of vibrator elements are arranged also in the slice direction like the array-type ultrasonic vibrator 100, it is difficult to sufficiently reduce the beam width in the slice direction by using only the acoustic lens. Therefore, in general, keeping the outputs of the vibrator elements at array end portions small (apodization) or reducing the beam width in the slice direction by adjusting a phase difference of ultrasonic vibrations of the vibrator elements are performed.

That apodization or phase adjustment can be realized by providing a wiring in the electrodes (corresponding to the upper electrode layer 103 and the lower electrode layer 104 in this embodiment) for each vibrator element and controlling the voltage and phase for each vibrator element. However, in a case where wirings are provided in all the individual vibrator elements, the number of wirings extending from the array-type ultrasonic vibrator increases, which results in complication of manufacturing processes and an increase in cost. In addition, a processor (multiplexer and the like) for controlling vibrations of each of the vibrator elements needs to be installed near the array-type ultrasonic vibrator, and it is difficult to utilize it in a case where the ultrasonic probe has a limited inner space, for example.

In contrast, in the array-type ultrasonic vibrator 100 according to this embodiment, the wirings are provided in a manner shown below. Beam focusing in the slice direction is realized while avoiding the problem as described above.

### [Regarding Wiring of Vibrator Element]

FIG. 5 is a schematic diagram showing an array of the vibrator elements 150 in the array-type ultrasonic vibrator 100. As shown in the figure, it is assumed that a row of the vibrator elements 150 in the slice direction (X direction) is an element row S. The array-type ultrasonic vibrator 100 is constituted by a plurality of element rows S. The number of vibrator elements 150 and the number of element rows S which constitute the element rows S are not particularly limited and both need to be plural.

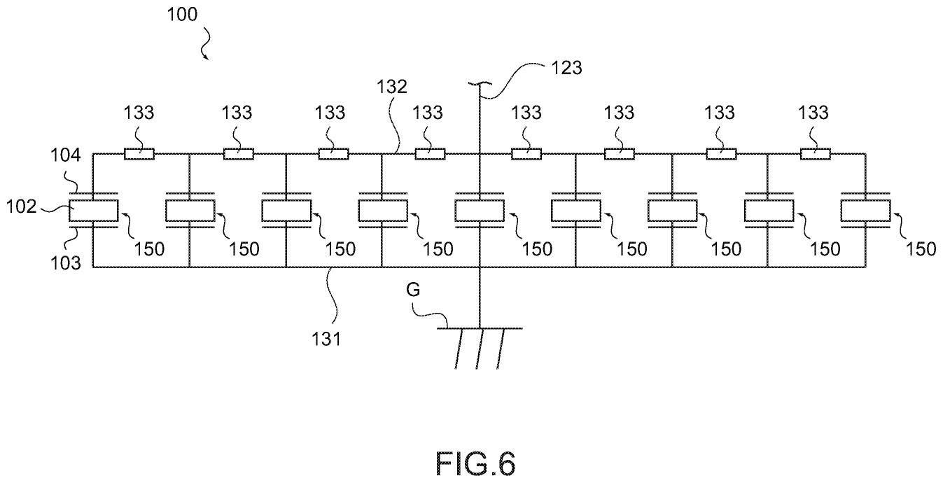

FIG. 6 is a schematic diagram showing an electric connection relationship of one of the element rows S. In the figure, the piezoelectric layers 102, the upper electrode layers 103, and the lower electrode layers 104 are schematically shown with respect to the respective vibrator elements 150.

As shown in the figure, the upper electrode layers 103 are connected to each other via a wiring 131 and are connected to a ground G. Further, the lower electrode layers 104 are connected to each other via a wiring 132 and are connected to the independent wiring 123. Resistors 133 are electrically connected between the vibrator elements 150 that constitute the element row S. It should be noted that the wiring 132 is realized by the wirings 125, the pad 124, and the wirings 122 in FIG. 4 and the resistors 133 are realized by the substrate built-in resistor 121. It should be noted that the resistance values of the respective resistors 133 may be identical to one another or may be different from one another. For example, the resistance values of the respective resistors 133 may be configured to be gradually higher from a center portion to end portions of the element row S.

The plurality of element rows S that constitute the array-type ultrasonic vibrator 100 each have the configuration shown in FIG. 6 and are not electrically connected between the element rows S. Therefore, in the array-type ultrasonic vibrator 100, the single independent wiring 123 is connected to each of the element rows S.

By providing the wirings in the vibrator elements 150 in the above-mentioned manner, the driving signal is supplied from the independent wiring 123 for each element row S and vibrations of the piezoelectric layers 102 are controlled for each element row S in the array-type ultrasonic vibrator 100.

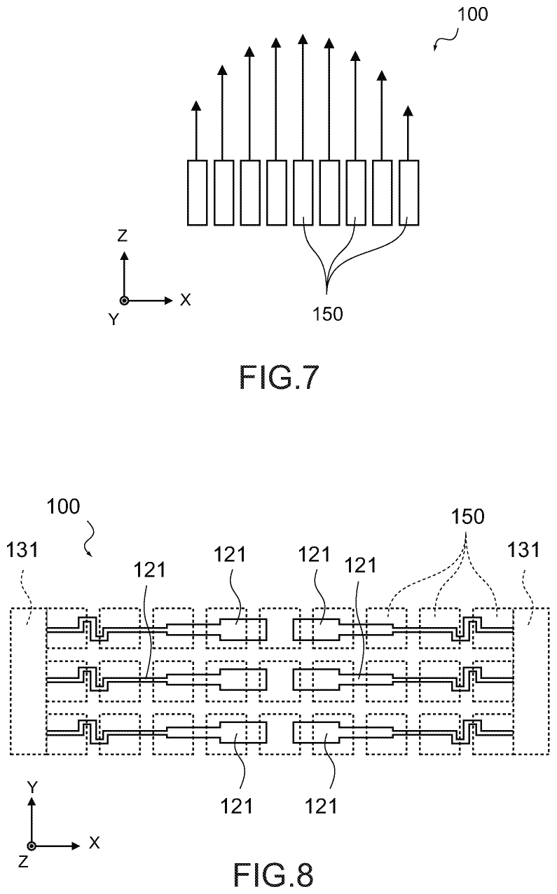

FIG. 7 is a schematic diagram showing an output of an ultrasonic wave radiated from the vibrator elements 150 in the array-type ultrasonic vibrator 100. In the figure, the magnitude of the output of the ultrasonic wave radiated from the vibrator element is indicated by the length of the arrow.

In each of the element rows S, a driving signal supplied from the independent wiring 123 is supplied to the vibrator elements 150 connected via the resistors 133 while the driving signal is being attenuated by the resistors 133. In this manner, as shown in FIG. 7, the output of the ultrasonic wave generated in the piezoelectric layers 102 becomes smaller with respect to the vibrator elements 150 closer to the end portions of each element row S.

As described above, the output of the ultrasonic wave is smaller at the end portions of each of the element rows S of the array-type ultrasonic vibrator 100. Therefore, the apodization is realized and the beam width in the slice direction is reduced.

Further, the independent wiring 123 is connected to each of the element rows S. Therefore, ultrasonic vibrations can be controlled in accordance with the driving signal in each element row S. That is, with respect to the azimuth direction, the apodization and the phase control can be realized in accordance with the driving signal.

As described above, in the array-type ultrasonic vibrator 100, with respect to the azimuth direction, the output of the ultrasonic wave can be adjusted in accordance with the driving signal for each element row S. With respect to the slice direction, the output of the ultrasonic wave is passively adjusted by the resistors 133. The wiring required in the array-type ultrasonic vibrator 100 is a single wiring for the single element rows S. Therefore, the number of wirings can be greatly reduced as compared to a case where the wiring is provided in each of the vibrator elements 150.

It should be noted that substantially a low pass filter is formed by the resistors 133 between the respective vibrator elements 150. However, a phase offset generated in each element row S can be reduced by being used at a cut-off frequency or less of the low pass filter.

### [Specific Structure of Wiring of Vibrator Element]

In the array-type ultrasonic vibrator 100, one capable of realizing the connection relationship as shown in FIG. 6 only needs to be provided and a specific structure is not particularly limited. However, as shown in FIG. 4, it is favorable to utilize the substrate built-in resistor 121.

FIG. 8 is a schematic diagram showing the substrate built-in resistors 121. FIG. 8 shows the vibrator elements 150, the substrate built-in resistors 121, and the wirings 131. The substrate built-in resistor 121 includes an electrically conductive material having a higher electric resistance. As shown in the figure, the substrate built-in resistor 121 is formed in such a shape that the width is gradually narrower toward the end portions of the element row S, that is, the electric resistance increases. The resistors 133 shown in FIG. 6 can be thus realized by the substrate built-in resistor 121.

It should be noted that the substrate built-in resistor 121 can be formed by depositing a Ni film, a NiCr film, a Ni—P element, a NiCrAlSi film, or the like by plating or sputtering and patterning it.

It should be noted that the resistors 133 can also be realized by utilizing an element other than the substrate built-in resistor 121 and, for example, a resistor compatible for a small-height embedded passive device (EPD) can also be utilized.

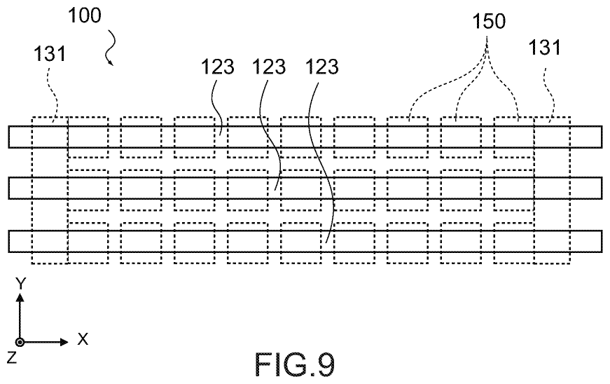

FIG. 9 is a schematic diagram showing the independent wirings 123. FIG. 9 shows the vibrator elements 150, the independent wirings 123, and the wirings 131. As shown in the figure, the independent wiring 123 are provided to extend in the slice direction (X direction).

### [Regarding Other Configurations of Array-Type Ultrasonic Vibrator]

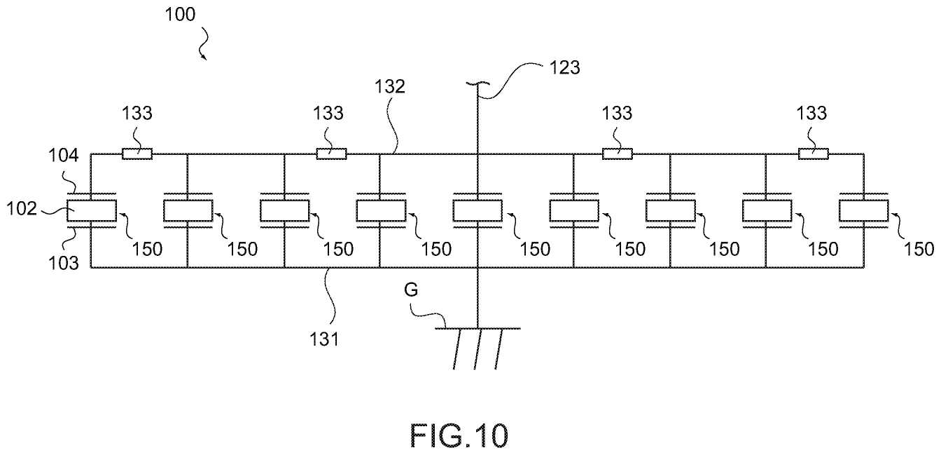

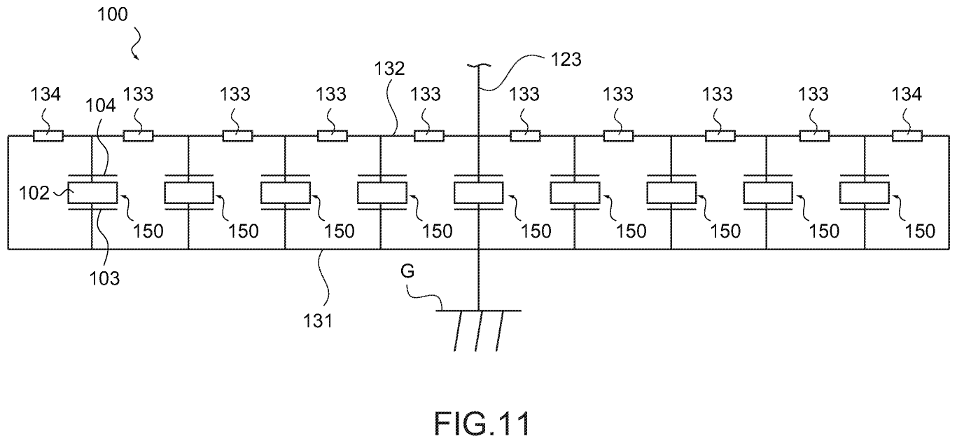

The configuration of the array-type ultrasonic vibrator 100 is not limited to the above. FIGS. 10 and 11 are schematic diagrams showing array-type ultrasonic vibrators 100 having other configurations.

As shown in FIG. 10, the resistors 133 do not need to be electrically connected between all the vibrator elements 150 and may be electrically connected between pairs of some of the vibrator elements 150. Also in this structure, the output of the ultrasonic wave at the end portions of the element row S becomes smaller. Therefore, the apodization is realized and the beam width in the slice direction is reduced.

Further, as shown in FIG. 11, the array-type ultrasonic vibrator 100 may include grounding resistors 134. The grounding resistors 134 are electrically connected between the vibrator elements 150 positioned at both the ends of the element row S and the ground G.

In such a configuration, there is a fear that electric charges accumulated at the end portions of the element row S remain also after the driving signal is input. In contrast, by providing the grounding resistors 134, it is possible to ensure an escape path for electric charges at the end portions of the element row S and to prevent electric charges from being accumulated at the end portions of the element row S.

It should be noted that if the resistance values of the grounding resistors 134 are too low, electric charges escape when the driving signal is input. On the other hand, if the resistance values of the grounding resistors 134 are too high, the provision of the grounding resistors 134 becomes a waste. Therefore, it is necessary to set a suitable resistance value. Further, the grounding resistors 134 may be provided with respect to the configuration in which the resistors 133 are provided between pairs of some of the vibrator elements 150, which is shown in FIG. 10.

### [Application Example of Array-Type Ultrasonic Vibrator]

An application example of the array-type ultrasonic vibrator 100 according to this embodiment will be described.

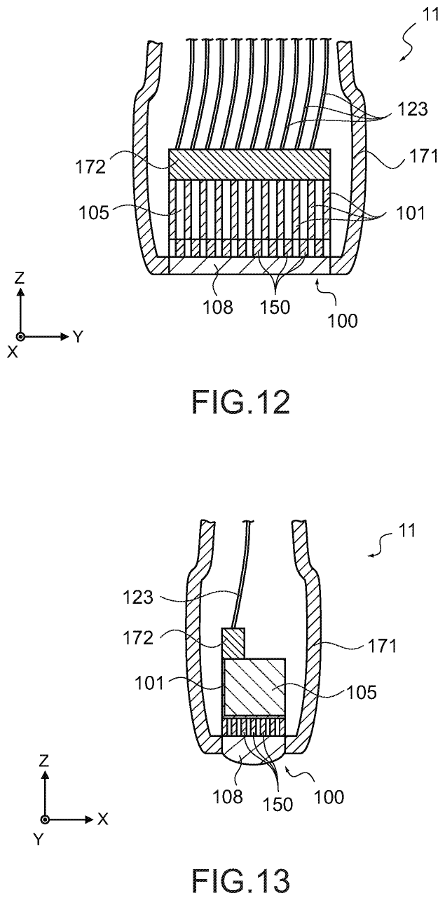

FIGS. 12 and 13 each are a schematic diagram of an ultrasonic probe 11 including the array-type ultrasonic vibrator 100. As shown in the figure, the ultrasonic probe 11 includes the array-type ultrasonic vibrator 100, a casing 171 that houses the array-type ultrasonic vibrator 100, and a wiring connection portion 172. The independent wirings 123 are connected to the wiring connection portion 172.

As described above, the array-type ultrasonic vibrator 100 only needs to connect the single independent wiring 123 to the single element row S. Therefore, the number of wirings can be greatly reduced as compared to a structure in which the wiring is provided in each of the vibrator elements 150.

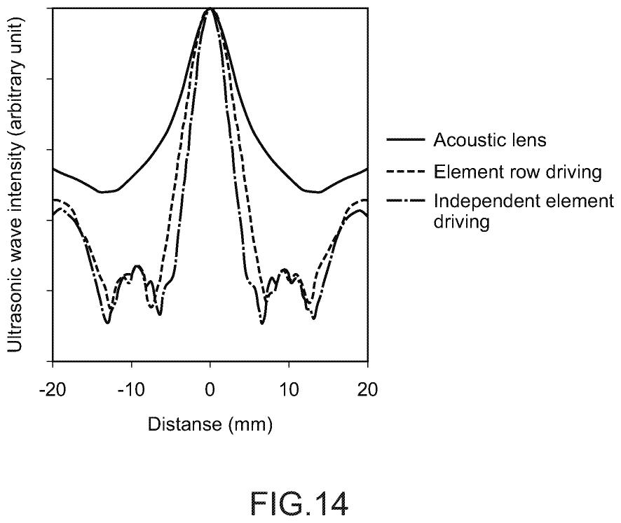

FIG. 14 is a graph showing simulation results of intensity of the ultrasonic wave radiated from the ultrasonic probe. FIG. 14 shows a beam profile at a measurement depth of 3.5 cm while the opening width of the ultrasonic probe in the slice direction (X direction) is set to 5 mm.

The “element row driving” in the figure indicates the intensity of the ultrasonic wave in a case where the apodization is carried out by utilizing the array-type ultrasonic vibrator 100 according to this embodiment. The “independent element driving” in the figure indicates the intensity of the ultrasonic wave in a case where the apodization is carried out by utilizing the array-type ultrasonic vibrator in which the wiring is connected to each of the vibrator elements. The “acoustic lens” in the figure indicates the intensity of the ultrasonic wave in a case where only the focusing effect by the acoustic lens is provided without utilizing the apodization.

In the “element row driving” according to this embodiment, side lobes (ultrasonic waves travelling in a direction deviated from a main radiation direction) are greatly reduced as compared to the “acoustic lens”, and a beam profile similar to that of the “independent element driving” is obtained.

Therefore, in the “element row driving”, an ultrasonic image excellent in the visibility can be generated having a dynamic range equivalent to that of the “independent element driving” while greatly reducing the number of wirings relative to the “independent element driving”.

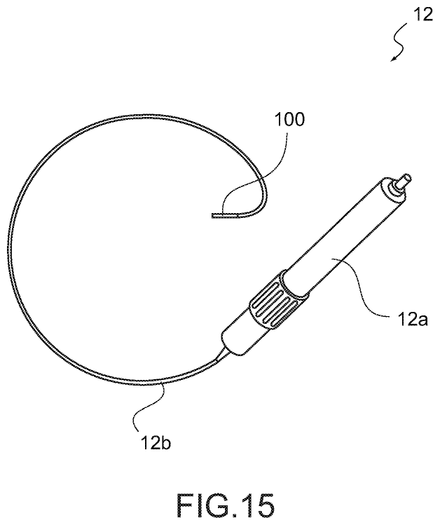

FIG. 15 is a schematic diagram of an ultrasonic catheter 12 including the array-type ultrasonic vibrator 100. The ultrasonic catheter 12 is an intracardiac ultrasonic catheter, for example. The ultrasonic catheter 12 includes a main body 12a and a catheter 12b. The array-type ultrasonic vibrator 100 is mounted on a distal end of the catheter 12b.

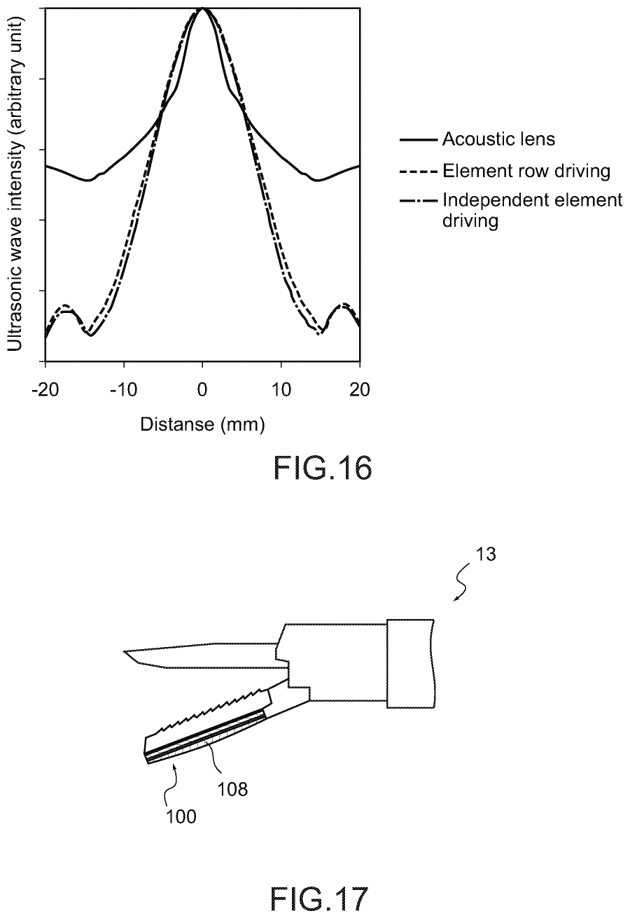

FIG. 16 is a graph showing simulation results of the intensity of the ultrasonic wave radiated from the ultrasonic catheter. FIG. 16 shows a beam profile in a case where the opening width of the ultrasonic probe in the slice direction (X direction) is set to 2 mm.

The “element row driving”, the “independent element driving”, and the “acoustic lens” have the same meanings as those described above. Also with the ultrasonic catheter, in the “element row driving”, the side lobes are greatly reduced as compared to the “acoustic lens”, and a beam profile similar to that of the “independent element driving” is obtained.

Further, regarding the ultrasonic catheter, it is necessary to bend and operate the catheter. Meanwhile, in the array-type ultrasonic vibrator in which the wiring is connected to each of the vibrator elements, the number of wirings inside the catheter increases, which becomes an obstacle to the operation. If the multiplexer and the like are mounted on the distal end portion of the catheter, the number of wirings inside the catheter can be reduced. However, it is not easy to do so because the mounting space of the distal end portion of the catheter is limited. Also in view of this, the array-type ultrasonic vibrator 100 which requires a smaller number of wirings is favorable.

FIG. 17 is a schematic diagram of a surgical instrument 13 including the array-type ultrasonic vibrator 100. The surgical instrument 13 is an incision tool or forceps and has a distal end portion on which the array-type ultrasonic vibrator 100 is mounted. In a case where the array-type ultrasonic vibrator is mounted on the surgical instrument in this manner, the housing space is smaller and the opening diameter is also smaller. Therefore, the dynamic range is deteriorated.

In contrast, the array-type ultrasonic vibrator 100 according to this embodiment has a higher dynamic range as described above. Therefore, the image quality of the ultrasonic diagnosis can be improved.

Further, the array-type ultrasonic vibrator 100 can be mounted on the ultrasonic probe together with a position sensor. The position sensor is a sensor that acquires the position of the ultrasonic probe and can be a magnetic sensor, for example. With such a configuration, on the basis of a relationship between a two-dimensional ultrasonic diagnosis image generated by the array-type ultrasonic vibrator 100 and the position of the ultrasonic probe, which is output from the position sensor, a three-dimensional volume image can be generated (see Japanese Patent Application Laid-open No. 2008-178500).

As described above, the array-type ultrasonic vibrator 100 according to this embodiment is capable of generating an ultrasonic diagnosis image having a high contrast. Therefore, a three-dimensional volume image having a high contrast can be generated by mounting the array-type ultrasonic vibrator 100 together with the position sensor.

### [Regarding Details of Resistance Value]

Details of the resistance values of the resistors 133 and the grounding resistors 134 in the array-type ultrasonic vibrator 100 including the resistors 133 and the grounding resistors 134, which are shown in FIG. 11, will be described.

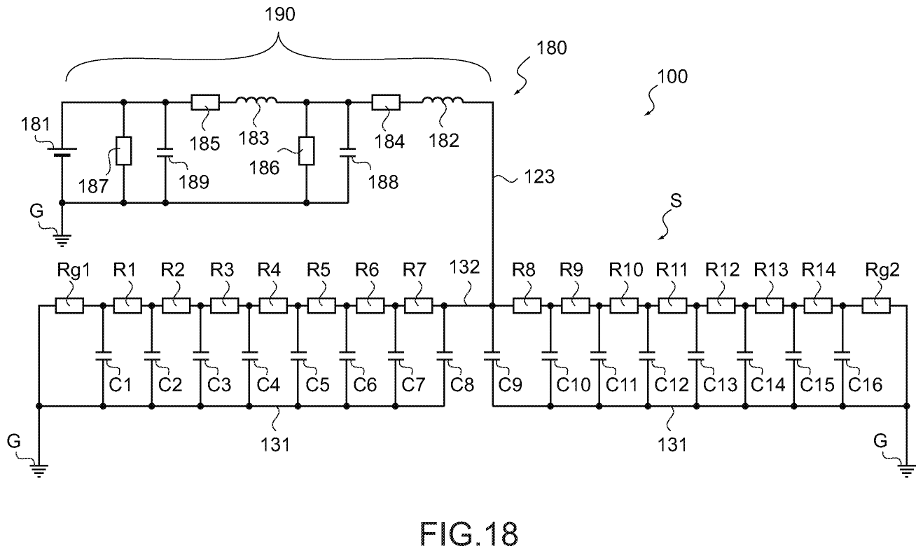

FIG. 18 is a schematic diagram showing an electric circuit configuration of each of the element rows S of the array-type ultrasonic vibrator 100. In the figure, capacitances formed by the respective vibrator elements 150 (see FIG. 11) are denoted by C1 to C16, resistances by the resistors 133 are denoted by R1 to R14, and resistances by the grounding resistors 134 are denoted by Rg1 and Rg2. It should be noted that the number of vibrator elements 150 and the number of resistors 133 are not limited to those shown in FIG. 18.

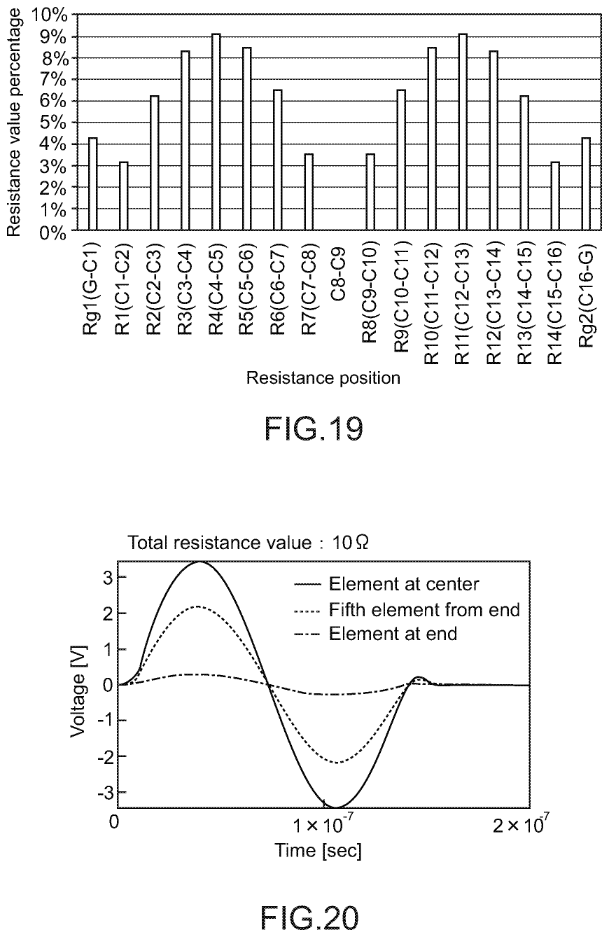

FIG. 19 is a graph showing the percentage of the resistance values of the resistors 133 and the grounding resistors 134 and is a graph showing the percentage of the resistance values of the resistors 133 and the grounding resistors 134, assuming that a total resistance value of the resistors 133 and the grounding resistors 134 is 100%. In the figure, the bracketed portion denotes a position of each of the resistors 133 and the grounding resistors 134.

Further, in FIG. 18, a configuration of a power supply circuit 180 connected to the element row S via the independent wiring 123 is shown. The power supply circuit 180 includes a driving power supply 181, an inductor 182, an inductor 183, a resistor 184, a resistor 185, a resistor 186, a resistor 187, a capacitor 188, and a capacitor 189. In addition, a wiring between the element row S and the driving power supply 181 is defined as a signal wiring 190. The signal wiring 190 is a coaxial cable. A resistance value thereof is 143 Ω, for example.

Here, it is favorable that, for the resistors 133 and the grounding resistors 134, a total resistance value (Rg1+Rg2+R1 + . . . +R14) of the resistors 133 and the grounding resistors 134 in the element row S is larger than a resistance value of the signal wiring 190.

In addition, provided that the frequency of the driving voltage of the driving power supply 181 is f [Hz], it is favorable that a product (hereinafter, RC) of the total resistance value of (R1+ . . . +R14) of the resistors 133 in the element row S and the total value (C1+ . . . +C16) of the capacitance of the vibrator elements 150 is smaller than ½ f.

FIGS. 20 to 24 are graphs showing simulation results of a voltage time history waveform with each of the vibrator elements 150 in the element row S. In each figure, the “element at center” indicates the vibrator element 150 corresponding to C8 or C9 in FIG. 18 and the “fifth element from end” indicates the vibrator element 150 corresponding to C5 or C12 in the figure. The “element at end” indicates the vibrator element 150 corresponding to C1 or C16 in the figure.

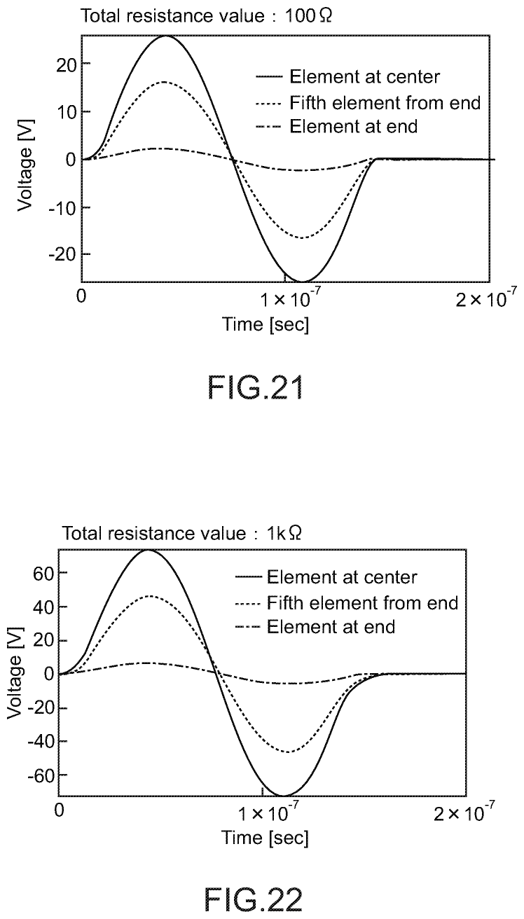

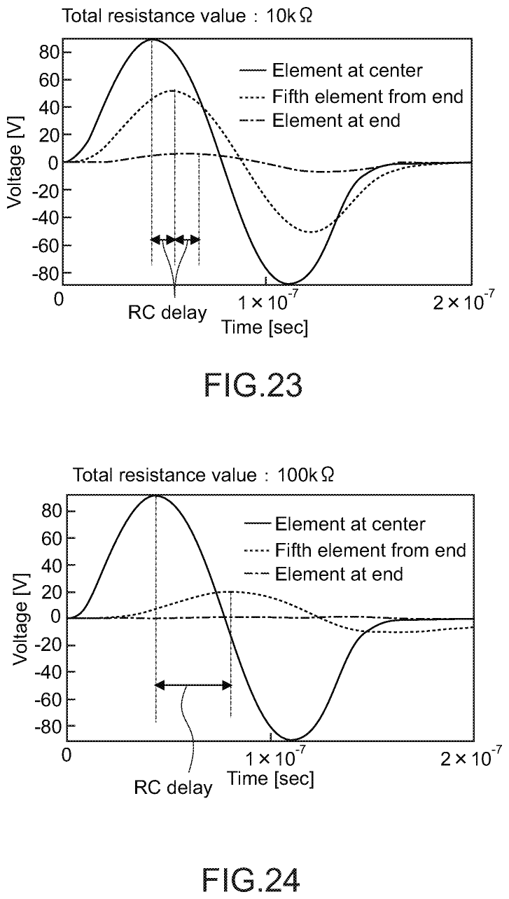

The total resistance value (hereinafter, total resistance value) of the resistors 133 and the grounding resistors 134 is 10 Ω, in FIG. 20, 100 Ω, in FIG. 21, 1 Ω in FIG. 22, 10 Ω, in FIGS. 23, and 100 Ω, in FIG. 24.

The analysis condition of the simulation is as follows: the vibrator element width in the azimuth direction (Y direction) is 90 μm; the array-type ultrasonic vibrator width (opening width) in the slice direction (X direction) is 5 mm; the number of vibrator elements in the slice direction (X direction) is 16; the thickness of the array-type ultrasonic vibrator is 120 μm; and the applied voltage waveform is 100 V, 7 MHz, Sin wave, 1 wave.

As shown in FIGS. 20 and 21, in a case where the total resistance value is smaller than the resistance value of the signal wiring 190 (143 Ω), a maximum voltage is about 3 V or 20 V with respect to an applied voltage of 100 V and a voltage drop of each of the vibrator elements 150 is too large.

On the other hand, as shown in FIGS. 22 to 24, in a case where the total resistance value is larger than the resistance value of the signal wiring 190 (143 Ω), a maximum voltage is about 60 V or 80 V with respect to an applied voltage of 100 V and a voltage drop of each of the vibrator elements 150 is not large.

Therefore, it is favorable that the total resistance value is larger than the resistance value of the signal wiring 190.

Further, the RC (product of the total resistance value of (R1+ . . . +R14) of the resistors 133 and the total value (C1+ . . . +C16) of the capacitance of the vibrator elements 150) is larger than ½ f, an RC delay (phase offset) is generated as shown in FIGS. 23 and 24.

Therefore, it is favorable that the RC is smaller than ½ f. It should be noted that in FIGS. 20 to 24, R is the “total resistance value” shown in the figure and C is 65.8 pF in any cases. While ½ f at 7 MHz is 71.4 nsec, the RC value is 0.658 pF under the condition of FIG. 20, 6.58 pF under the condition of FIG. 21, 65.8 pF under the condition of FIG. 22, 658 pF under the condition of FIGS. 23, and 6.58 nF under the condition of FIG. 24.

For this reason, it is favorable that the total resistance value is larger than the resistance value of the signal wiring 190 and it is favorable that the RC is smaller than ½ f. In a case where those conditions are satisfied as shown in FIG. 22 (total resistance value: 1 kΩ), a voltage drop of each of the vibrator elements 150 is small and generation of the RC delay is also prevented.

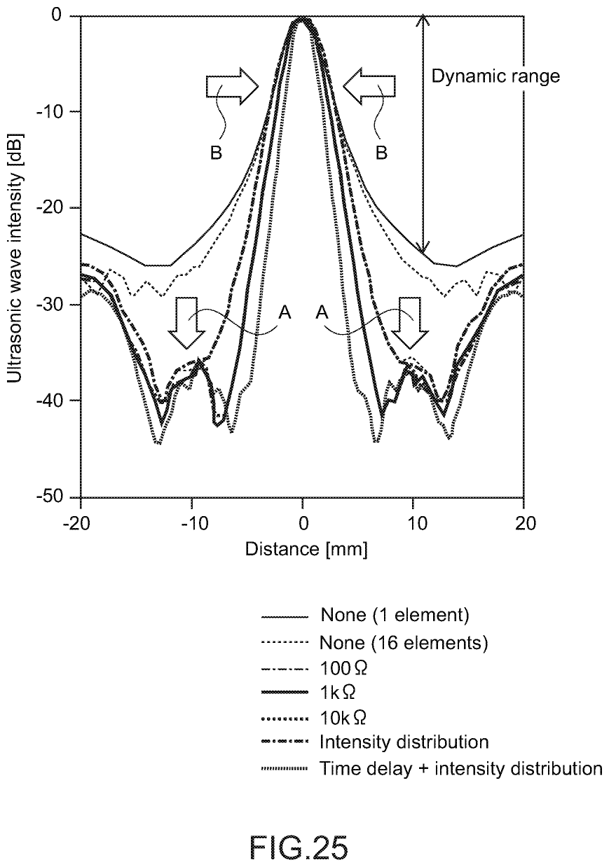

FIG. 25 shows simulation results of a sound pressure beam profile of the array-type ultrasonic vibrator 100 in a case where the width (opening width) in the X direction is 5 mm. Here, the focal distance is 35 mm.

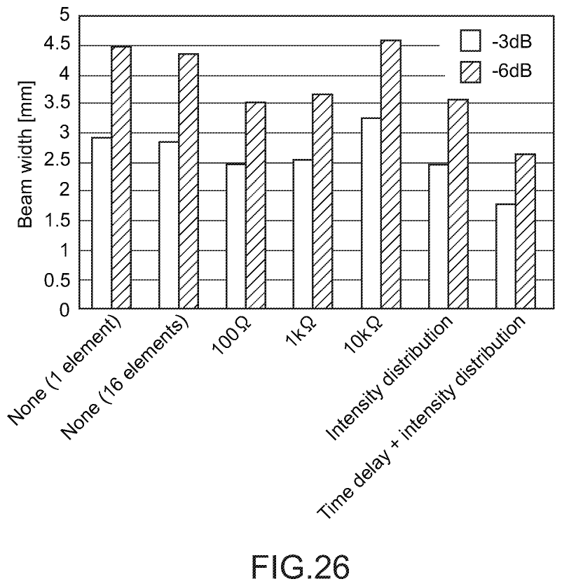

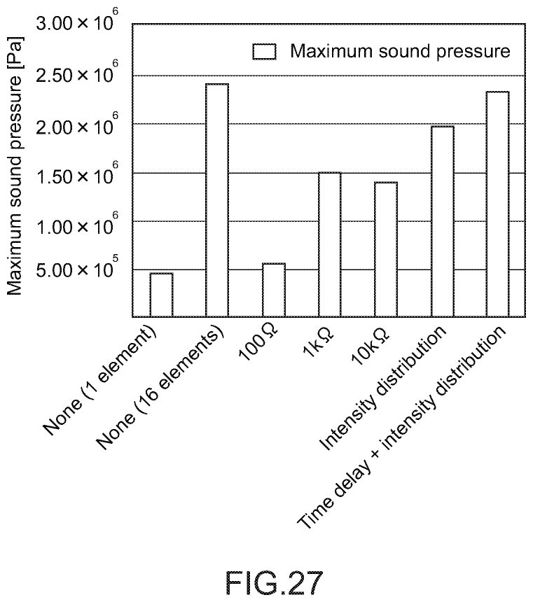

FIG. 26 is a graph showing a beam width (width at the time of lowering by −3 dB and −6 dB) with the sound pressure beam profile shown in FIG. 25. FIG. 27 is a graph showing a dynamic range with the sound pressure beam profile shown in FIG. 25.

As shown in those figures, in a case of the opening width 5 mm, the effects of the improvement of the dynamic range (in FIG. 25, the arrow A) and the reduction of the beam width (in FIG. 25, the arrow B) are obtained when the total resistance value is 1 kΩ.

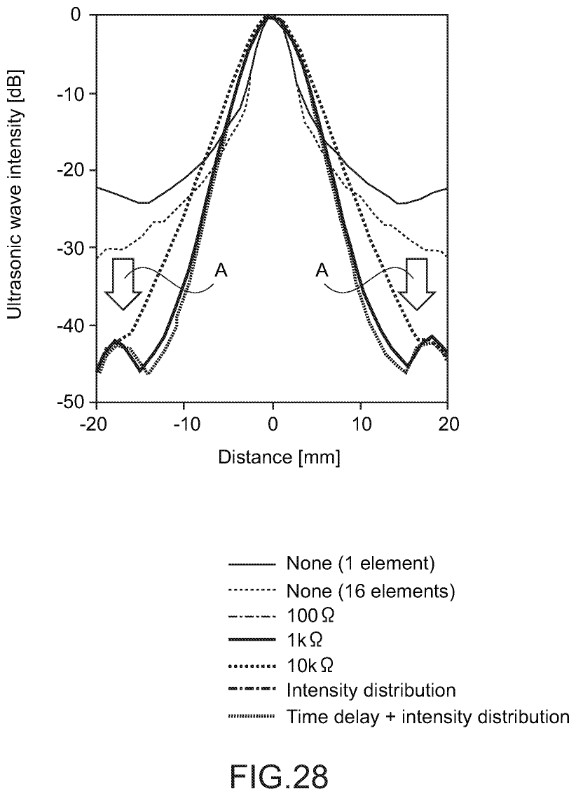

FIG. 28 shows other simulation results of the sound pressure beam profile of the array-type ultrasonic vibrator 100 in a case where the width (opening width) in the X direction is 2 mm. Here, the focal distance is 35 mm.

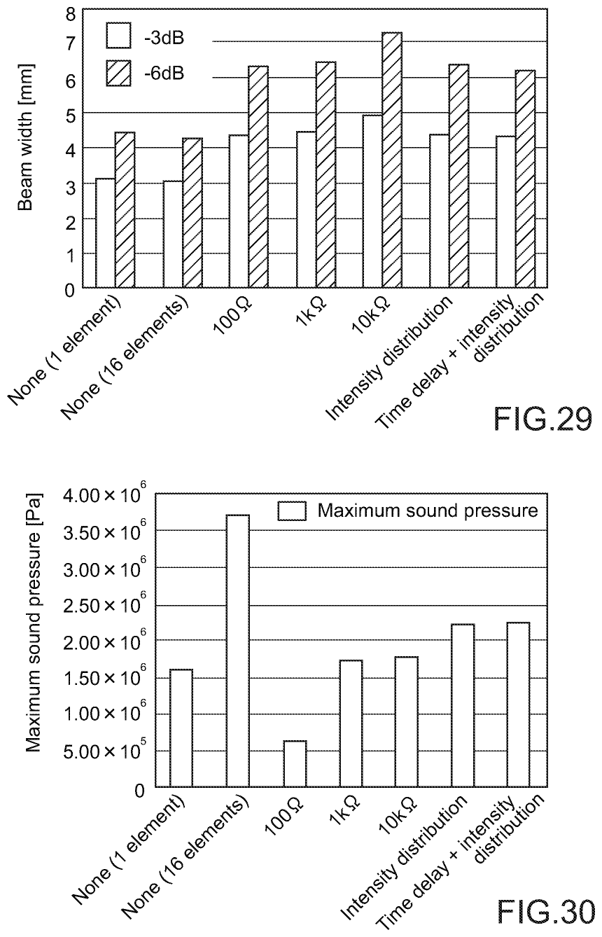

FIG. 29 is a graph showing a beam width (width at the time of lowering by −3 dB and −6 dB) with the sound pressure beam profile shown in FIG. 28. FIG. 30 is a graph showing the dynamic range with the sound pressure beam profile shown in FIG. 28.

As shown in those figures, in a case where the opening width is 2 mm, the effect of the improvement of the dynamic range (in FIG. 28, the arrow A) is obtained when the total resistance value is 1 kΩ.

In this manner, in a case where the total resistance value is larger than the resistance value of the signal wiring 190 and the RC is smaller than ½ f, the effects of the improvement of the dynamic range of the array-type ultrasonic vibrator 100 and the reduction of the beam width are obtained.

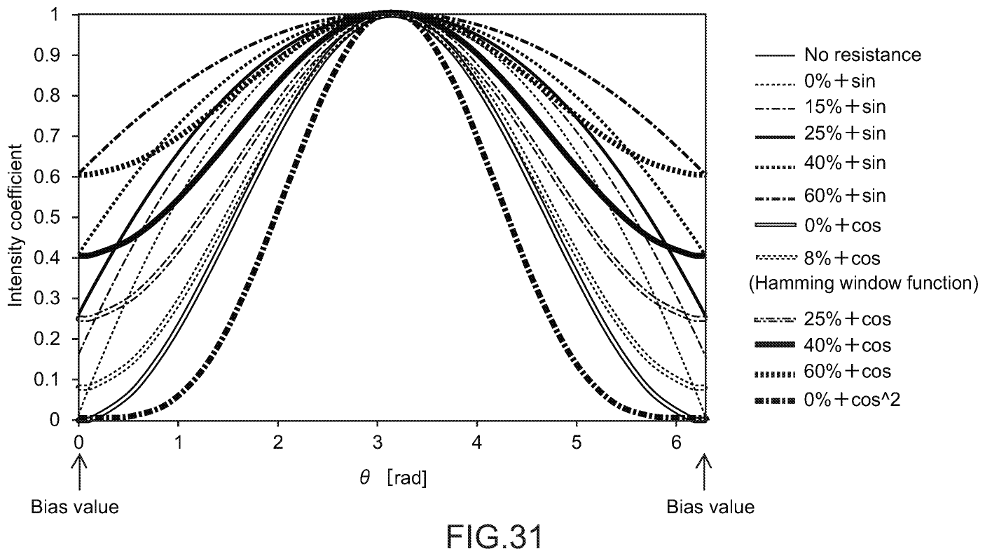

Further, in each of the element rows S, it is favorable that the resistance values of the grounding resistors 134 (Rg1 and Rg2) are smaller than the total resistance value (R1+ . . . +R14) of the resistors 133. FIG. 31 is a graph showing the apodization intensity distribution in each of the element rows S. As the bias value (intensity coefficient at the end) becomes smaller, the dynamic range becomes larger.

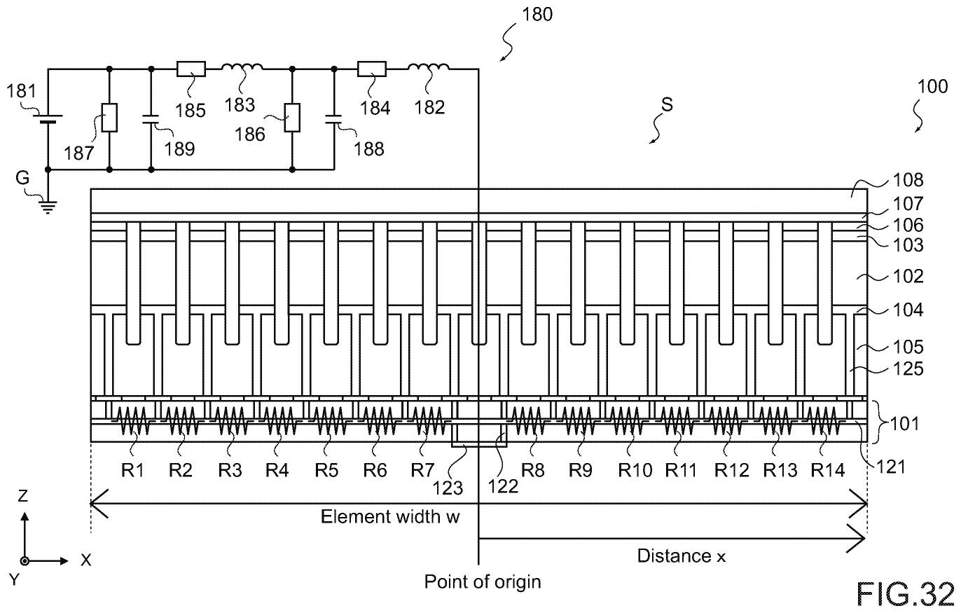

More specifically, as shown in FIG. 32, provided that the number of vibrator elements 150 of the element row S is n, the width of the element row S in the slice direction (X direction) is w, and the center in the slice direction is a point of origin, and also provided that the distance from the point of origin in the slice direction is x, a peal value of a voltage waveform which is generated in a kth vibrator element 150 from the end is Vk due to the driving voltage of the driving power supply 181, and a maximum value of Vk (1≤k≤n) is Vmax, it is favorable that the distribution of Vk is a distribution according to a hamming window function shown in (Expression 1) below.

Vk/Vmax=0.54+0.46 cos(2π*x/w) (Expression 1)

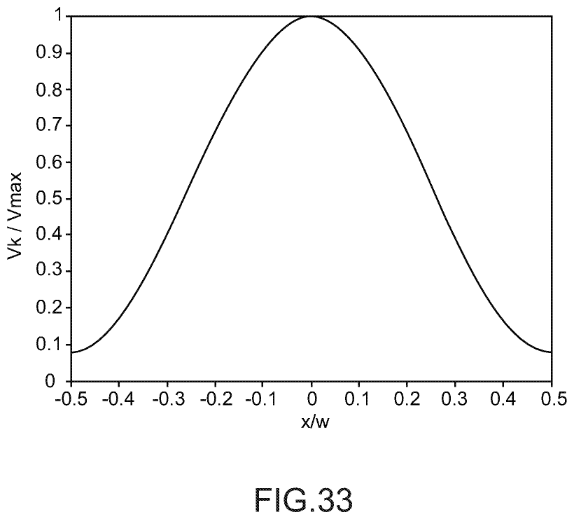

FIG. 33 is a graph showing the hamming window function shown in (Expression 1) above.

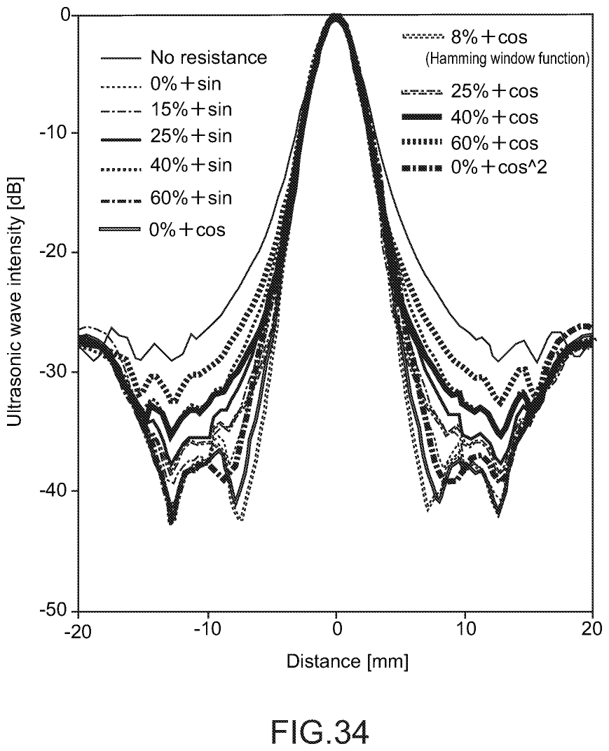

FIG. 34 shows simulation results of the sound pressure beam profile of the array-type ultrasonic vibrator 100 in a case where the width (opening width) in the X direction is 5 mm. Here, the focal distance is 35 mm.

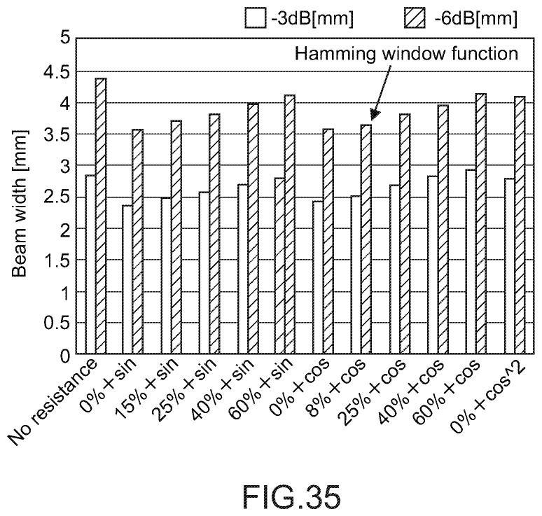

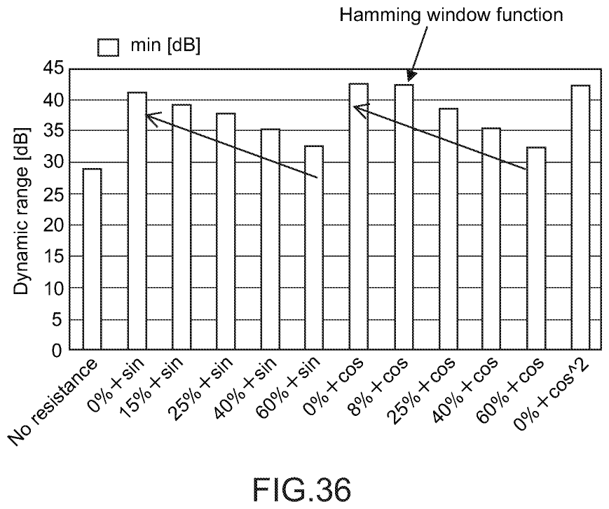

FIG. 35 is a graph showing a beam width (width at the time of lowering by −3 dB and −6 dB) with the sound pressure beam profile shown in FIG. 34. FIG. 36 is a graph showing a dynamic range with the sound pressure beam profile shown in FIG. 34.

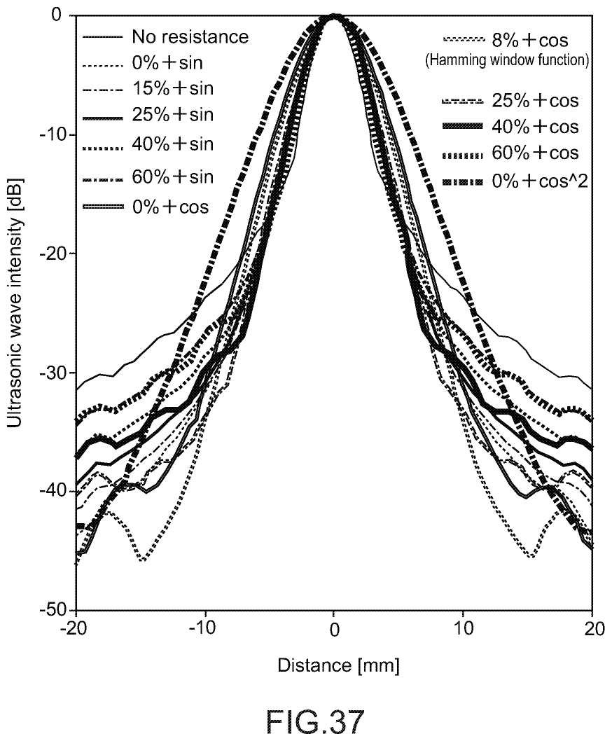

FIG. 37 shows simulation results of the sound pressure beam profile of the array-type ultrasonic vibrator 100 in a case where the width (opening width) in the X direction is 2 mm. Here, the focal distance is 35 mm.

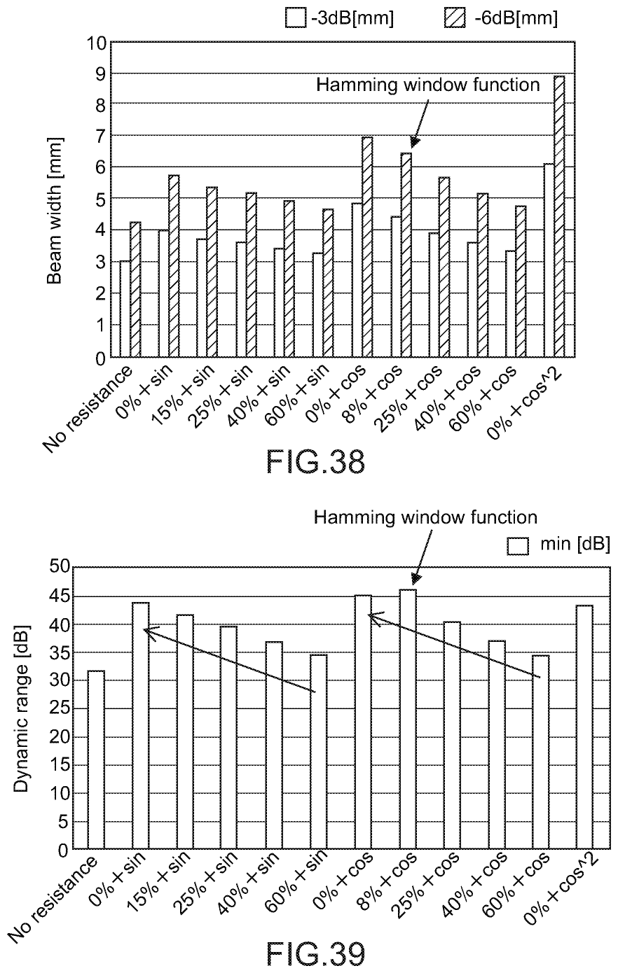

FIG. 38 is a graph showing a beam width (width at the time of lowering by −3 dB and −6 dB) with the sound pressure beam profile shown in FIG. 37. FIG. 39 is a graph showing a dynamic range with the sound pressure beam profile shown in FIG. 37.

As shown in those figures, in a case where the intensity distribution function is the hamming window function, the dynamic range becomes larger, which is favorable.

As described above, by providing the configuration in which the resistance values of the grounding resistors 134 are smaller than the total resistance value of the resistors 133 and the apodization intensity distribution is a distribution according to the hamming window function, the effect of the improvement of the dynamic range of the array-type ultrasonic vibrator 100 is obtained.

It should be noted that the present technology may also take the following configurations.

(1) An array-type ultrasonic vibrator, including:
a vibrator array in which ultrasonic vibrator elements constitute a two-dimensional array including a plurality of element rows in which a plurality of ultrasonic vibrator elements are arranged in a slice direction; and
a resistor electrically connected between a pair of arbitrary ultrasonic vibrator elements in the element row.

(2) The array-type ultrasonic vibrator according to (1), in which
the resistor is electrically connected between all ultrasonic vibration elements in the element row.

(3) The array-type ultrasonic vibrator according to (1) or (2), further including
a grounding resistor connected between an ultrasonic vibration element at an end of the element row and a ground.

(4) The array-type ultrasonic vibrator according to any one of (1) to (3), in which
the vibrator array includes a substrate that supports the ultrasonic vibrator elements, and
the resistor is implemented on a surface of the substrate or inside the substrate.

(5) The array-type ultrasonic vibrator according to any one of (1) to (4), in which
the ultrasonic vibrator elements are connected to a wiring for driving the ultrasonic vibrator elements in each of the element rows.

(6) An ultrasonic probe, including
an array-type ultrasonic vibrator including
   a vibrator array in which ultrasonic vibrator elements constitute a two-dimensional array including a plurality of element rows in which a plurality of ultrasonic vibrator elements are arranged in a slice direction, and
   a resistor electrically connected between a pair of arbitrary ultrasonic vibration elements in the element row.

(7) An ultrasonic catheter, including
an array-type ultrasonic vibrator including
   a vibrator array in which ultrasonic vibrator elements constitute a two-dimensional array including a plurality of element rows in which a plurality of ultrasonic vibrator elements are arranged in a slice direction, and
   a resistor electrically connected between a pair of arbitrary ultrasonic vibration elements in the element row.

(8) A hand-held surgical instrument, including:
an array-type ultrasonic vibrator including
   a vibrator array in which ultrasonic vibrator elements constitute a two-dimensional array including a plurality of element rows in which a plurality of ultrasonic vibrator elements are arranged in a slice direction, and
   a resistor electrically connected between a pair of arbitrary ultrasonic vibration elements in the element row.

(9) A medical apparatus, including:
an array-type ultrasonic vibrator including
   a vibrator array in which ultrasonic vibrator elements constitute a two-dimensional array including a plurality of element rows in which a plurality of ultrasonic vibrator elements are arranged in a slice direction, and
   a resistor electrically connected between a pair of arbitrary ultrasonic vibration elements in the element row; and
a position sensor that detects a position of the array-type ultrasonic vibrator.

(10) The medical apparatus according to (9), in which
an ultrasonic volume image is generated on the basis of outputs of the array-type ultrasonic vibrator and the position sensor.

(11) The array-type ultrasonic vibrator according to any one of (3) to (5), in which
in each of the element rows, a total resistance value of the resistor and the grounding resistor is larger than a resistance value of a signal wiring that connects the ultrasonic vibrator element to a driving power supply.

(12) The array-type ultrasonic vibrator according to any one of (3) to (5) and (11), in which
in each of the element rows, provided that a frequency of a driving voltage of the ultrasonic vibrator element is f [Hz], a product of a total resistance value of the resistor and a total value of capacitance of the ultrasonic vibrator elements is smaller than ½ f.

(13) The array-type ultrasonic vibrator according to any one of (3) to (5) and (11) and (12), in which
in each of the element rows, a resistance value of the grounding resistor is smaller than a total resistance value of the resistor.

### REFERENCE SIGNS LIST

11 ultrasonic probe

12 ultrasonic catheter

13 surgical instrument

100 array-type ultrasonic vibrator

101 substrate

102 piezoelectric layer

103 upper electrode layer

104 lower electrode layer

105 backing layer

106, 107 acoustic matching lens

107 acoustic matching lens

108 acoustic lens

121 substrate built-in resistor

123 independent wiring

133 resistor

134 grounding resistor

150 vibrator element

1. An array-type ultrasonic vibrator, comprising:
a vibrator array in which ultrasonic vibrator elements constitute a two-dimensional array including a plurality of element rows in which a plurality of ultrasonic vibrator elements are arranged in a slice direction; and
a resistor electrically connected between a pair of arbitrary ultrasonic vibrator elements in the element row.

2. The array-type ultrasonic vibrator according to claim 1, wherein the resistor is electrically connected between all ultrasonic vibration elements in the element row.

3. The array-type ultrasonic vibrator according to claim 1, further comprising
a grounding resistor connected between an ultrasonic vibration element at an end of the element row and a ground.

4. The array-type ultrasonic vibrator according to claim 1, wherein
the vibrator array includes a substrate that supports the ultrasonic vibrator elements, and
the resistor is implemented on a surface of the substrate or inside the substrate.

5. The array-type ultrasonic vibrator according to claim 1, wherein
the ultrasonic vibrator elements are connected to a wiring for driving the ultrasonic vibrator elements in each of the element rows.

6. An ultrasonic probe, comprising
an array-type ultrasonic vibrator including
a vibrator array in which ultrasonic vibrator elements constitute a two-dimensional array including a plurality of element rows in which a plurality of ultrasonic vibrator elements are arranged in a slice direction, and
a resistor electrically connected between a pair of arbitrary ultrasonic vibration elements in the element row.

7. An ultrasonic catheter, comprising
an array-type ultrasonic vibrator including
a vibrator array in which ultrasonic vibrator elements constitute a two-dimensional array including a plurality of element rows in which a plurality of ultrasonic vibrator elements are arranged in a slice direction, and
a resistor electrically connected between a pair of arbitrary ultrasonic vibration elements in the element row.

8. A hand-held surgical instrument, comprising:
an array-type ultrasonic vibrator including
a vibrator array in which ultrasonic vibrator elements constitute a two-dimensional array including a plurality of element rows in which a plurality of ultrasonic vibrator elements are arranged in a slice direction, and
a resistor electrically connected between a pair of arbitrary ultrasonic vibration elements in the element row.

9. A medical apparatus, comprising:
an array-type ultrasonic vibrator including
a vibrator array in which ultrasonic vibrator elements constitute a two-dimensional array including a plurality of element rows in which a plurality of ultrasonic vibrator elements are arranged in a slice direction, and
a resistor electrically connected between a pair of arbitrary ultrasonic vibration elements in the element row; and
a position sensor that detects a position of the array-type ultrasonic vibrator.

10. The medical apparatus according to claim 9, wherein
an ultrasonic volume image is generated on a basis of outputs of the array-type ultrasonic vibrator and the position sensor.

11. The array-type ultrasonic vibrator according to claim 3, wherein in each of the element rows, a total resistance value of the resistor and the grounding resistor is larger than a resistance value of a signal wiring that connects the ultrasonic vibrator element to a driving power supply.

12. The array-type ultrasonic vibrator according to claim 3, wherein in each of the element rows, provided that a frequency of a driving voltage of the ultrasonic vibrator element is f [Hz], a product of a total resistance value of the resistor and a total value of capacitance of the ultrasonic vibrator elements is smaller than ½ f.

13. The array-type ultrasonic vibrator according to claim 3, wherein in each of the element rows, a resistance value of the grounding resistor is smaller than a total resistance value of the resistor.
